(19) United States
(12) Patent Application Publication
(10) Pub. No.: US 2003/0225101 A1
(43) Pub. Date: Dec. 4, 2003

(54) Method of treating retinal occlusion with sildenafil

(76) Inventor: Ronald L. Scheman, Washington, DC (US)

Correspondence Address: Mr. L. Ron Scheman, 5002 50th Place, N.W., Washington, DC, 20016-4380, US

(21) Appl. No.: 10/157,893

(22) Filed: May 31, 2002

(51) Int. Cl. A61K 31/519

(52) U.S. Cl. 514/252.16

(57) ABSTRACT

A symptom of an ocular circulatory condition in a human being is treated by administering an effective amount of 1-[4-ethoxy-3-(6,7-dihydro-1-methyl-7-oxo-3-propyl-1H-pyrazolo-[4,3-d]pyrimidin-5-yl)phenylsulphonyl]-4-methylpiperazine or a pharmaceutically acceptable salt thereof to the human being.

### FIELD OF THE INVENTION

The present invention relates generally to a method of treating ocular circulatory conditions. More particularly, the invention relates to treating central retinal vein occlusion with an effective amount of sildenafil or a pharmaceutically acceptable salt thereof.

### BACKGROUND OF THE INVENTION

Ocular circulatory conditions are a widespread problem. Estimates of the frequency of only one type of ocular circulatory condition, central retinal vein occlusion (CRVO), are approximately 60,000 cases per year in the United States alone.

Ocular circulatory conditions refer to both intraocular blood circulation and aqueous humor circulation conditions. Intraocular blood circulation relates to blood flow to the eye through the arterial system, through the capillary system and from the eye through the venous system. Aqueous humor circulation relates to flow of the clear watery fluid between the cornea and the iris of the eye that helps the cornea keep its shape.

A specific ocular circulation condition that is particularly disturbing is retinal vein occlusion (RVO). RVO can occur suddenly and without notice and may cause blindness. Although the disease has been known since the 1800s and a large volume of literature has been published on the subject, its management is still ill-understood and controversial. Currently there is no effective treatment for RVO. Sohan Singh Hayreh, MD, MS, PhD, DSc, FRCS, FRCOphth, Central Retinal Vein Occlusion, (The University of Iowa, December 2000) [retrieved on Aug. 3, 2001].

RVO appears equally in males and females and is more common in the elderly, often with a history of systemic ailments such as diabetes and hypertension. Typically, the afflicted person is older than the age of 50, with the majority of persons between the ages of 50 and 70.

RVO affects the circulation of blood within the eye. The retina of the eye receives most of its blood supply from the retinal vessels which supply the inner two thirds of the retina. Damage to retinal blood vessels resulting in closure of retinal capillaries occurs in several disease processes including RVO. Particularly, the retinal artery supplies blood to the retina and the blood flows through the retinal arterioles, capillaries and then through branch retinal veins that drain into the central retinal vein. A blockage in one of these veins leads to RVO. The blockage of a branch retinal vein and the central retinal vein are distinguished as branch retinal vein occlusion (BRVO) and CRVO, respectively.

It is possible to further separate RVO into a more elaborate framework. The two divisions are ischemic RVO—the creation of a condition of deficiency of oxygenation—and non-ischemic RVO—the lack of such a condition. Classification of RVO into non-ischemic and ischemic RVO is essential because typically non-ischemic RVO is a comparatively benign disease, while ischemic RVO is a seriously blinding disease. The two types have very different effects, presentations, symptoms, etiologies, treatments, and ultimate outcomes.

The blockages of both the branch retinal vein and the central retinal vein cause back pressure. This back pressure has a variety of consequences. The increased venous pressure can lead to hemorrhaging, exudation and decreased blood flow in the area of the retina drained by the particular section of vein that is occluded.

In CRVO, the leaking and bleeding from the capillaries can cause swelling in the macula—a portion of the retina that absorbs short wavelength light—and in the optic disc—a spot on the retina where nerve bundles are attached—both which can lead to the loss of vision, as well as, blurred vision, eye pain, and the like.

The decreased blood flow in retina can cause angiogenesis or neovascularization—the creation of new blood vessels. When the blood flow is sufficiently restricted an ischemic condition can occur. Ischemic RVO results in the release of one or more angiogenic factors that stimulate neovascularization, the most feared complication. Neovascularization can occur on the retina, on the outer surface of the vitreous, and on the iris, all with similar results.

Retinal neovascularization can bleed or cause severe scarring of the retina. The new vessels break through the internal limiting membrane that lines the inner surface of the retina and grow along the outer surface of the vitreous. They recruit many other cells and produce sheets of vessels, cells, and extracellular matrices that exert traction on the retina. The neovasuclarization can also often lead to detachment of the retina and severe loss of vision.

Ischemic RVO and the resulting neovascularization can lead to increased eye pressure—neovascular glaucoma. Neovascular glaucoma is caused by abnormal blood vessels growing on the iris and leads to pressure build-up inside the eye.

These conditions can be detected by a variety of methods. First, although non-ischemic RVO may develop much more subtly than ischemic RVO, the disease is characterized by a sudden onset.

Second, vision loss—either blurred or missing area of vision, depending on the vein that is blocked—is an extreme symptom of all the preceding conditions. The reduction of one's Visual field also indicates an onset of the condition. Markedly decreased visual acuity tends to be more common in ischemic RVO. eMedicine.com reported that a study demonstrated that visual acuity of 20/200 or better was seen in 80.9% of non-ischemic and only 6.7% of ischemic RVO. Richard J Spitz, MD, Retinal Vein Occlusion, 2 eMedicine J. No. 5 (May 22, 2001).

Third, the retinal hemorrhages associated with RVO are typically present after a few days and may be detectable with direct ophthalmoscopy. Peripheral hemorrhages tend to be the first to appear and may only be visible with indirect ophthalmoscopy. Retinal hemorrhages may also be found incidentally on fundoscopic examination without visual complaint. Alternatively, patients may complain of increasing blurry vision, usually worse in the morning upon waking and improving throughout the day. Diurnal variation in blood pressure is thought to account for this effect. Relative nighttime hypotension may lead to decreased retinal vein blood flow, which may cause the daily variation in vision. The history may be progressive, because of increasing macular edema. Similarly, the blurry vision may be preceded by episodes of amaurosis fugax.

Fourth, neovascularization and retinal occlusions may also be diagnosed using a test called a fluorescein angiogram. This involves injecting a dye into an arm vein and taking photographs of the retina as the dye circulates through it. This test will give an ophthalmologist information about the health of the circulation inside a patient's eye.

Finally, the effects of RVO and its complications can be detected in the following ways: observing the presence of cotton wool spots that represent deeper focal retinal ischemia and its associated scarring and angiogenesis; viewing dilated, tortuous retinal veins caused by the backpressure associated with RVO; and measuring raised intraocular pressure.

Generally, the causes of RVO can be classified into local disease processes and systemic disease processes. The local disease processes include trauma, open and closed angle glaucoma, orbital abscesses, orbital tumors, and vascular hypertension. In contrast, the systemic disease processes include arteriosclerosis, diabetes mellitus, hypertension, hyperviscosity, coagulation disorders (antithrombin III deficiency, antiphospholipid antibodies, activated protein C resistance), dehydration, cocaine use, blood dyscrasias (leukemia, lymphoma, sickle cell anemia), vasculitides, renal insufficiency, dysproteinemias.

The most frustrating aspect of RVO is that there is currently no effective treatment. Many investigators have studied this problem and attempted treatments without success. Periodically there are claims of success made for one medication or another, but no treatment has proved effective under scientific testing conditions. Dr. Michael Pinnolis, Retinal Vein Occlusion, Harvard Vanguard Medical Services.

Attempts at treating this condition are numerous. Several attempts have been directed to the occlusion itself. For example, U.S. Pat. No. 6,268,359 (issued Jul. 31, 2001) discloses a pharmaceutical composition for preventing or treating visual function disorders caused by ocular circulatory disorders (for example, retinal vein occlusion) whose active component is a vasopressin antagonist. The antagonist may be a number of different compounds including benzazepine derivatives.

Doctors have also administered fibrinolytic or thrombolytic agents, such as TPA and streptokinase, to dissolve the preformed thrombus in the central retinal vein. U.S. Pat. No. 4,795,423 discloses an example of this practice, However, the administration of these drugs can cause a significant amount of bleeding and have had their effectiveness questioned.

Anti-coagulants and anti-platelet agents have also been prescribed. It is suggested that this treatment can be harmful because it may promote hemorrhaging in the eye. Sohan Singh Hayreh, MD, MS, PhD, DSc, FRCS, FRCOphth, Central Retinal Vein Occlusion, (The University of Iowa, December 2000).

Further, U.S. Pat. No. 6,245,038 claims a method for treating CRVO by withdrawing blood from the patient, treating the blood by plasmapheresis techniques and re-infusing the treated blood. The object of the plasmapheresis is to control the blood's viscosity.

Additionally, U.S. Pat. No. 6,090,847 discloses that EP₂-receptor agonists are potent neuroprotective agents, especially for the eyes of mammals. The art identifies (.+-.) trans-2-[-4(1-hydroxyhexyl) phenyl]-5-oxocyclopentaneheptanoicacid as one such antagonist. Two other methods known to have been used to treat RVO are surgical decompression of the central retinal vein and laser-induced chorioretinal venous anastomosis (for treatment of non-ischemic CRVO). Dr. Michael Pinnolis, Retinal Vein Occlusion, Harvard Vanguard Medical Services.

Although, there are some treatments directed towards the occlusion itself, most of the treatments for RVO concentrate on the conditions created by the occlusion—1) edema in the eye, 2) angiogenesis or neovascularization, and 3) increased eye pressure.

U.S. Pat. No. 6,086,869 treats retinal edema, which is associated with RVO, by administering interferon. For many years it was thought that laser treatment played a useful role in treating macular edema. However, in recent years, at least one article cites a major scientific study that concluded that laser treatment was of no benefit in resolving or improving macular edema. Dr. Michael Pinnolis, Retinal Vein Occlusion, Harvard Vanguard Medical Services.

Ophthalmologists also utilize systemic corticosterioids in an effort to reduce the macular edema that is associated with the condition. Sohan Singh Hayreh, MD, MS, PhD, DSc, FRCS, FRCOphth, Central Retinal Vein Occlusion, (The University of Iowa, December 2000).

Methods to treat neovascularization may or may not involve the administration of a compound to the patient. One method to treat neovascularization that does not involve the administration of a compound is panretinal photocoagulation and laser-induced chorioretinal venous anastomosis.

Several different classes of compounds have been introduced as anti-neovascularization agents. First, tyrosine kinase inhibitors have been disclosed to prevent angiogenesis and neovascularization. U.S. Pat. No. 6,228,871 (the '871 patent) discloses 1) that vascular endothelial growth factor (VEGF) binds the high affinity membrane-spanning tyrosine kinase receptor KDR, 2) that cell culture and gene knockout experiments indicate that each receptor contributes to different aspects of angiogenesis, 3) that KDR mediates the mitogenic function of VEGF and 4) that inhibiting KDR modulates the level of mitogenic VEGF activity. The '871 patent claims compounds to inhibit tyrosine kinase enzymes. U.S. Pat. No. 6,265,403 and U.S. Pat. No. 6,235,741 also employ tyrosine kinase inhibitors. Further, several different types of inhibitors have been disclosed. U.S. Pat. No. 6,245,759 claims pyrazolo pyrimidinyl compounds. U.S. Pat. No. 6,204,011 discusses an isolated nucleic acid molecule that encodes a novel human receptor type tyrosine kinase gene, KDR, that is expressed on human endothelial cells. U.S. Pat. No. 6,162,804 discloses benzinidazole compounds.

Second, other patents disclose compounds that treat VEGF stimulated endothelial cell growth via inhibiting of the beta enzyme of Protein Kinase C (PKC). U.S. Pat. No. 6,114,320 (the '320 patent) uses compounds such as bis-indolylmaleimide or a macrocyclic bis-indolylmaleimide to counteract the effect of VEGF. The '320 patent also discloses that VEGF is thought to play a major part in mediating active intraocular neovascularization in patients with ischemic retinal diseases.

Third, compounds that inhibit the cell matrices and the cell-cell adhesion that are required for angiogenesis have been disclosed. U.S. Pat. No. 6,214,834 discusses amine and ammonium prodrugs and novel nonpeptide compounds which bind to integrin receptors thereby altering cell-matrix and cell-cell adhesion processes. U.S. Pat. No. 6,153,628 and U.S. Pat. No. 6,130,231 disclose other compounds that antagonize integrin receptors, i.e. 1,3,4-thiadiazoles and 1,3,4 oxadiazoles and fused heterocycles.

Fourth, U.S. Pat. No. 6,124,259 (the '259 patent) claims a method for treating ophthalmic disorders associated with excess insulin-like growth factor (IFG). IGF can create fibroproliferation within the eye. The '259 patent claims compounds that use Insulin-like Growth Factor Binding Protein (IGFBP) to treat diseases.

Other attempts have been made to prevent blood vessel growth. Specifically, U.S. Pat. No. 5,994,341 discloses the use of Anti-Invasive Factor, retinoic acids and derivatives thereof, and paclitaxel to prevent angiogenesis; U.S. Pat. No. 5,994,292 discusses interferon-inducible protein 10 as a potent inhibitor of angiogenesis; U.S. Pat. No. 6,214,819 discloses the use of staurosporine to limit neovascularization; and U.S. Pat. No. 6,217,895 employs an implanted release device to deliver corticosterioids that do not have the typical side effect of increasing intraocular pressure, yet still treat neovascularization.

The most serious effect of CRVO is neovascular glaucoma and therefore many of the treatments for CRVO are directed towards neovascular glaucoma. Several types of compounds have been suggested to treat this complication. First, U.S. Pat. No. 6,258,844 and U.S. Pat. No. 6,124,353 disclose the use of prostaglandin, more specifically, omega chain modified prostaglandins and 8-epi prostaglandins of specific formulas, respectively.

Second, the use of cyclopentane 1-hydroxy alkyl and alkenyl-2-one or 2-hydroxy derivatives as ocular hypotensives has been disclosed. A few patents that discuss these compounds include, U.S. Pat. No. 6,248,783; U.S. Pat. No. 6,124,344; U.S. Pat. No. 6,096,902; U.S. Pat. No. 6,037,364; and U.S. Pat. No. 5,972,991. U.S. Pat. No. 5,990,138 discloses similar compounds, 7-[5-hydroxy-2-(hydroxyhydrocarbyl or heteroatom-substituted hydroxy hydrocarbyl)-3-hydroxycyclopentyl(enyl)] heptanoic or heptenoic acids and derivatives of said acids, wherein one or more of said hydroxy groups are replaced by an ether group. Also, U.S. Pat. No. 6,248,773, U.S. Pat. No. 6,160,129 and U.S. Pat. No. 6,204,287 relate analogous compounds, cyclopentane heptan(ene)oic acid, 2-heteroarylalkenyl derivatives. U.S. Pat. No. 6,204,287 describes these compounds as derivatives of F-type prostaglandins.

Third, compounds useful as thromboxane ligands for reducing ocular pressure have been disclosed. More specifically, U.S. Pat. No. 6,090,845 and U.S. Pat. No. 6,017,953 discuss bicyclic carboxylic acids wherein the bicyclic rings may be hydrocarbyl or oxyhydrocarbyl, thromboxane ligands, as a method for treating ocular hypertension.

Fourth, U.S. Pat. No. 6,194,415, and U.S. Pat. No. 6,248,741 disclose compounds containing 2-imidazolin-2ylamino quinoxalines as components of treatments for high intraocular pressure.

Finally, acetazolarnide can be administered in an effort to reduce the glaucomatic pressure; however, this substance does not work in all patients. Sohan Singh Hayreh, MD, MS, PhD, DSc, FRCS, FRCOphth, Central Retinal Vein Occlusion, (The University of Iowa, December 2000).

Practitioners believe that neovascular glaucoma can be treated with laser therapy, if the condition is detected early. Studies have been cited to show that the success for laser therapy is higher if the treatment is not given preventatively. That is, it is thought best to wait until the problem has actually begun, rather than to treat ahead of time in anticipation of problems. Unfortunately this means a patient with a central retinal vein occlusion will need to be examined carefully about every 4-6 weeks during the first six months after diagnosis in order to catch the problem on time. Dr. Michael Pinnolis, Retinal Vein Occlusion, Harvard Vanguard Medical Services. This level of medical attention requires both the doctor and the patient to expend a burdensome amount of time to achieve beneficial results and requires that a large amount of money be spent to treat the condition.

There are suggestions that the belief that lowering the intra-ocular pressure improves retinal blood flow on which treatments for ocular hypotensive therapy are based is erroneous.

None of the above approaches has had any real success. The above treatments do not resolve the occlusion, rather the blockage tends to disappear on its own. Although, this may take anywhere from a few months to a few years. Sohan Singh Hayreh, MD, MS, PhD, DSc, FRCS, FRCOphth, Central Retinal Vein Occlusion, (The University of Iowa, December 2000).

Unfortunately, RVO is a very frustrating problem and final vision is often not very good. The visual outcome is not improved by any treatment since there is no way to alter the natural progression of the vein occlusion itself. Thus, it is important for the physician to monitor the situation carefully and to administer appropriate laser treatment if complications arise. However, the vision may or may not return on its own.

Patients who experience a branch vein occlusion often notice a gradual improvement in their vision as the hemorrhage resolves. Recovery from a central vein occlusion is much less likely since it affects the macula.

As is apparent from the foregoing, many problems remain to be solved in the treatment of RVO, especially in the use of inhibitory drugs to effectively treat RVO. It would be highly advantageous to develop new methods for inhibiting the reduction in vision that is associated with RVO. In addition, the delivery of compounds that produce inhibitory effects of the RVO would be advantageous. Local administration of such compounds would also be useful in the treatment of other conditions where the target cell population is accessible by such administration.

### BRIEF SUMMARY OF THE INVENTION

In one respect, the invention pertains to a method of treating a symptom of an ocular circulatory condition in a human being. In this method, an effective amount of 1-[4-ethoxy-3-(6,7-dihydro-1-methyl-7-oxo-3-propyl-1H-pyrazolo-[4,3-d]pyrimidin-5-yl)phenylsulphonyl]-4-methylpiperazine or a pharmaceutically acceptable salt thereof for treating the symptom of the ocular circulatory condition is administered to the human being.

In another respect, the invention relates to a method of treating retinal vein occlusion. In this method, a safe and effective amount of a pharmaceutical composition is administered to a patient in need thereof. Additionally, the pharmaceutical composition is sildenafil; or a pharmaceutically acceptable salt thereof.

In yet another respect, the invention pertains to a method of preventing the progression of retinal vein occlusion. In this method, a safe and effective amount of a pharmaceutical composition is administered to a patient in need thereof. Additionally, the pharmaceutical composition is sildenafil; or a pharmaceutically acceptable salt thereof.

In yet another respect, the invention relates to a method of preventing the progression of a symptom of retinal vein occlusion in a human being. In this method, an effective amount of 1-[4-ethoxy-3-(6,7-dihydro-1-methyl-7-oxo-3-propyl-1H-pyrazolo-[4,3-d]pyrimidin-5-yl)phenylsulphonyl]-4-methylpiperazine or a pharmaceutically acceptable salt thereof for treating the symptom of retinal vein occlusion is administered to the human being.

### DETAILED DESCRIPTION

The present invention is a method for treating the symptoms associated with treating ocular circulatory conditions by the administration of Viagra™ (Pfizer Labs, N.Y.) as an effective RVO therapeutic agent. Further the present invention describes the prophylactic and or therapeutic administration of Viagra™ in patients exhibiting RVO disease and RVO associated diseases. The details of the invention are described below.

Viagra™ is presently a commercial medication for impotence. According to the manufacturer, in addition to the main ingredient, sildenafil citrate, each tablet also contains the following ingredients: microcrystalline cellulose, anhydrous dibasic calcium phosphate, croscannellose sodium, magnesium stearate, hydroxypropyl methylcellulose, titanium dioxide, lactose, triacetin, and FD&C Blue #2 aluminum lake.

Viagra™ is formulated in tablets equivalent to 25 mg, 50 mg and 100 mg of sildenafil citrate for oral administration. Sildenafil citrate can be absorbed orally from suspensions, drops, caplets and chewable and non-chewable tablet formulations.

Sildenafil in Viagra™ is reported to be rapidly absorbed after oral administration, with absolute bioavailability of about 40%. Its pharmacokinetics are dose-proportional over the recommended dose range. Based on the Viagra™ manufacturer's product literature, maximum observed plasma concentrations are reached within 30 to 120 minutes (median 60 minutes) of oral dosing in the fasted state. When the Viagra™ formulation is taken with a high fat meal, the rate of absorption is reduced, with a mean delay in Tmax of 60 minutes and mean reduction in Cmax of 29%. The mean steady state volume of distribution for sildenafil is reportedly 105 L, indicating distribution into the tissues. Based upon reported measurements of sildenafil in the semen of healthy volunteers 90 minutes after dosing, less than 0.001% of the administered dose appeared in the semen of the patients.

The therapeutically effective dosage of sildenafil in Viagra™ was determined by in vivo studies. The minimum effective dosage of sildenafil in Viagra™ is contemplated to be 25 mg, while the maximum recommended dosage is 100 mg per day. Such doses may be administered in a single administration or may be administered over a variety of time intervals during a particular treatment regimen.

The structure and properties of sildenafil are well known. The hydrophobic chemical has a molecular weight of 474.6. Sildenafil and its pharmaceutically acceptable salts are described in U.S. Pat. No. 5,250,534, entitled “Pyrazolopyrimidinone Antianginal Agents”, the disclosure of which is hereby incorporated by reference.

Sildenafil is 1-[4-ethoxy-3-(6,7-dihydro-1-methyl-7-oxo-3-propyl-1H-pyrazolo[4,3-d]pyrim idin-5-yl)phenylsulphonyl]-4-methylpiperazine. It is a substituted guanine analog and has the following structure:

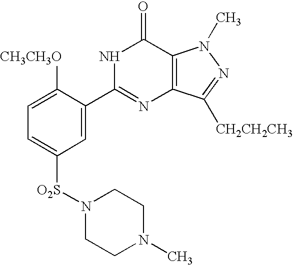

The generic name sildenafil represents a free base and the present invention includes its pharmaceutically acceptable salts, such as hydrochloride, hydrobromide, hydroiodide, nitrate, sulfate, bisulfate, phosphate, acid phosphate, acetate, lactate, citrate, acid citrate, tartrate, bitartrate, salicylate, succinate, maleate, gluconate, methane sulfate, ethane sulfate, methane sulfonate (mesylate), benzenesulfonate (besylate), toluene sulfonate, p-toluene sulfonate salts, organo-carboxylic acids and inorganic acids. Preferably, the salt form is sildenafil citrate, which has a solubility in water of 3.5 mg/ml.

Sildenafil has been shown to inhibit the activity of phosphodiesterase (PDE). The body has several types of PDEs. Studies in vitro have shown that sildenafil is selective for PDE5. Its effect is more potent on PDE5 than on other known PDEs. Sildenafil's reactivity is greater than 80 times that for PDE1 (a cGMP-hydrolyzing isozyme found predominantly in the brain, kidney, and smooth muscle), greater than 1000 times that for PDE2 (isozyme found predominantly in the adrenal cortex) and PDE4 (found predominantly in the brain and lung lymphocytes), greater than approximately 4000 times that for PDE3 (found predominantly in smooth muscles, platelets, and cardiac tissue) and about 10 times that for PDE6 (an enzyme found in the photoreceptors of the human retina).

Its reactivity with PDE5 is the factor that has prompted sildenafil citrate to be marketed as a treatment of erectile dysfunction. The physiological mechanism of erection of the penis involves release of nitric oxide (NO) in the corpus cavemosum during sexual stimulation. NO then activates the enzyme guanylate clase, which results in increased levels of cyclic guanosine monophosphate (cGMP), producing smooth muscle relaxation in the corpus cavernosum and allowing inflow of blood. Sildenafil has no direct relaxant effect on isolated corpus cavernosum, but enhances the effect of NO by inhibiting PDE5, which is responsible for degradation of cGMP in the corpus cavernosum. When sexual stimulation causes local release of NO, inhibition of PDE5 by sildenafil causes increased levels of cGMP in the corpus cavernosum, resulting in smooth muscle relaxation and inflow of blood to the corpus cavernosum. Sildenafil at recommended doses has no effect in the absence of sexual stimulation.

Several other uses for sildenafil have been suggested. For example, U.S. Pat. No. 6,242,444 anticipates the treatment of angina, hypertension, congestive heart failure, peripheral vascular disease, arteriosclerosis, stroke, bronchitis, asthma, allergic rhinitis and glaucoma. EP0463756 indicates that the class of compounds associated with sildenafil is useful for conditions of reduced blood vessel patency, post percutaneous transluminal coronary angioplasty, allergic asthma and diseases characterized by gut motility, i.e. irritable bowel syndrome. The patent also describes these compounds as platelet anti-aggregators and anti-vasopastic and vasodilatory agents.

Finally, U.S. Pat. No. 6,075,028 discloses sildenafil as a treatment for Tourette's syndrome and other central nervous system disorders, including attention deficit disorder.

The American Heart Association has reported that the adverse effects of sildenafil reflect its pharmacological activity of inhibition of PDE5 in various tissues and can be broadly classified into 4 major adverse reactions:

1. Vasodilatory effects resulting in headache (16%), flushing (10%), and rhinitis (4%) (the latter presumably as a result of hyperemia of nasal mucosa where PDE5 is present). Dizziness (2%), hypotension (<2%), and postural hypotension (<2%) have been reported rarely and occur at a similar rate in sildenafil- and placebo-treated patients.

2. Visual abnormalities resulting in blue-green color-tinged vision, increased perception of light, and blurred vision (3%), especially at higher doses.

3. Gastrointestinal effects resulting in dyspepsia and burning sensation from reflux due to relaxation of lower esophageal sphincter (7%).

4. Musculoskeletal effects resulting in myalgias, especially with multiple daily doses. No treatment-related changes in serum creatine kinase or electromyogram have been observed, however. There is no obvious pharmacological explanation for this effect.

Melvin D. Cheitlin, MD, Uses of Sildenafil (Viagra™) in Patients With Cardiovascular Disease (American College of Cardiology and American Heart Association, Inc., 1999).

The vasodilatory effects of sildenafil have caused the American Heart Association to issue warnings concerning its possible effects on the heart. Reported cardiovascular side effects in the normal healthy population are typically minor and associated with vasodilatation (i.e., headache, flushing, and small decreases in systolic and diastolic blood pressures). The approximately 4000-fold greater selectivity for PDE5 over PDE3 is important because inhibitors of PDE3 (the isozyme involved in regulation of cardiac contractility), such as milrinone, vesnarinone, and enoximone, that have been used in patients with heart failure are generally associated with increased incidence of cardiac arrhythmias and other serious side effects.

However, although their incidence is small, serious cardiovascular events, including significant hypotension, can occur in certain populations at risk. Most at risk are individuals who are concurrently taking organic nitrates. Organic nitrate preparations are commonly prescribed to manage the symptoms of angina pectoris. The co-administration of nitrates and sildenafil significantly increases the risk of potentially life-threatening hypotension. For this reason, sildenafil is contraindicated for patients taking other organic nitrates.

Although definitive evidence is currently lacking, it is possible that a precipitous reduction in blood pressure with nitrate use may occur over the initial 24 hours after a dose of sildenafil. Thus, the American Heart Association recommends that patients who experience an acute cardiac ischemic event and who have taken sildenafil within the past 24 hours, should avoid the administration of nitrates. It also suggests that in the event that nitrates are given, especially within this critical time interval, it is essential to have the capability to support the patient with fluid resuscitation and α-adrenergic agonists if needed.

Other patients in whom the use of sildenafil is potentially hazardous include those with active coronary ischemia; those with congestive heart failure and borderline low blood volume and low blood pressure status; those with complicated, multi-drug, anti-hypertensive therapy regimens; and those taking medications that may affect the metabolic clearance of sildenafil. Once again, the American Heart Association advises that until adequate studies are done in these subgroups of patients, sildenafil should be prescribed with caution.

The American Heart Association has further reported that the transient visual abnormalities (mostly color-tinged [blue-green] vision, increased perception of light, and blurred vision) that have been reported in patients taking sildenafil are mostly at high oral doses (>100 mg). These visual effects appear to be related to the weaker inhibiting action of sildenafil on PDE6, which regulates signal transduction pathways in the retinal photoreceptors. When light hits the retina a conformational change occurs that allows transducin, a protein, to become active. The transducin then activates PDE6 by binding to one of its subunits. The PDE6 then hydrolyzes cGMP, causing the closure of ion channels in the photoreceptors, reduced influx of sodium and calcium, and hyper-polarization of the receptor cell. Concerns have been expressed that the effect of sildenafil on vision may cause some longer-term sequelae on retinal function, but as yet there is no evidence for or against this.

According to a German study, healthy volunteers who took a single dose of sildenafil (Viagra™) showed changes in retinal function on sophisticated testing one hour after the medication was taken, but these changes disappeared within 6 hours. The test showed no change in the clarity or range of the men's vision, and no changes in their color vision or in their intraocular pressure. But the researchers did find that electroretinogram tests showed a slight loss in light sensitivity an hour after taking the drug. This corresponded with blood tests that found the highest concentration of the drug in blood drawn one hour after the drug was taken. Several hours later, the results of this test were again normal.

In an editorial, Dr, Eberhart Zrenner of Germany's University Eye Hospital in Tubigen writes that these study findings are “not alarming” in healthy patients with normal retinas. He notes that the effect of Viagra™ on the retina demonstrated that “only a very weak loss in light sensitivity . . . similar to the light-absorbing effect of a car windshield.” In addition, scientists have found that inhibiting PDE6 in lower animal models for long periods of time leads to photoreceptor cell degeneration.

In a separate effort, a University of Maryland Medical Center neuro-ophthalmologist, Howard Pomeranz, M.D., says he has discovered that, for a few men, taking sildenafil may result in going blind. He believes that the drug can cut off blood flow to the optic nerve. In a very short time, measured in minutes, that damages the nerve and causes permanent loss of vision in the affected eye. The technical name for the condition is ischemic optic neuropathy. Pomeranz describes it as “a stroke of the eye.” He has yet to define the risk of experiencing ischemic optic neuropathy after taking sildenafil. Pomeranz believes it is very small, but that it should not be overlooked.

The American Heart Association recommends that patients with inherited disorders of retinal PDE6, such as retinitis pigmentosa, should be not be administered sildenafil unless extreme caution is taken (Pfizer, unpublished data). In addition, the information guide to physicians packaged with Viagra™ was amended to include a warning that they should be cautious about prescribing it for patients with the inherited condition retinitis pigmentosa, a rare condition that causes progressive loss of sight. The insert says there is no safety information on the administration of the drug to people with the disease, which is at present incurable.

While some scientists believe that administering sildenafil has negative implications on ocular acuity and health, unexpectedly, it is believed than the administration of Viagra™ has resulted in a reduction in the symptoms associated with ocular circulatory conditions, such as RVO, CRVO, and the like. More specifically, it is believed that the administration of sildenafil or a salt thereof is effective in relieving or preventing the progression of RVO and CRVO. Preferably, a sildenafil salt is dispensed in tablet form. Sildenafil or its salt is administered in an amount and at a frequency that is effective in relieving the symptoms of RVO. Preferably, the sildenafil compound is administered in single doses on a regular basis, about 25 mg up to about 100 mg. Most preferably, about 50 mg of sildenafil or its salt is administered on a regular basis.

### EXAMPLE

A 68 year-old male patient with failing vision was first diagnosed with CRVO on Apr. 23, 1999. The patient had a history of keratoconus since age 16 and had worn hard contact lenses for 40 years. The patient was sent to various opthalogical specialist at the National Institutes of Health, the National Eye Institute, the Retina Group of Washington. The consensus among the various specialist indicated untreatable CRVO.

On Apr. 23, 1999 the patient was examined and the patient reported awakening a month earlier and suddenly noticed a large black spot in front of the central vision, left eye. The patient began taking 1 or 2 aspirins a day for the problem. Upon examination, the following was noted:

EXAMINATION:

| | |
|---|---|
| VISUAL | OD: 20/200 uncorrected; 20/30+ corrected; |
| ACUITY: | pinhole 20/20. |
| | OS: 20/400 uncorrected; 20/200 corrected; pinhole |
| | unimprovable. |
| VISUAL | OU: Full to count finger confrontation. |
| FIELD: | |
| EXTERNAL: | Normal adnexa. |
| PUPILS: | OD: Reactive to better amplitude of excursion |
| | than left. |
| | OS: Afferent pupil defect. |
| MOTILITY: | Ductions full, versions normal. |
| APPLANATION | OD: 14. |
| TENSIONS: | OS: 11. |
| SLIT LAMP | OD: Focal comeal nebulae; keratoconus; anterior |
| BIO- | chamber formed with slight temporal shallowing; |
| MICROSCOPY | normal iris; +2 brunescent nuclear sclerosis. |
| | OS: As OD without nebulae. |
| DILATED | OD: Disc pink, cup/disc ratio 0.2. Mild retinal |
| EXTENDED | vein engorgement and tortuosity . Normal macula, |
| OPHTHAL- | periphery. |
| MOSCOPY: | OS: Disc pink, slightly hyperemic; retinal venous |
| | engorgement and tortuosity; scattered retinal |
| | hemorrhages throughout the fundus; posterior |
| | pole edema. |
| DIAGNOSIS: | Central retinal vein occlusion, left eye, with macular |
| | edema.; possible incipient central retinal vein |
| | occlusion, right eye, versus chronic engorgement. |

It was further noted that there was no proven treatment specifically for central retinal vein occlusion, but the patient should would be worked up for underlying systemic etiologies. Considering the patient's age, unilateral central retinal vein occlusion is most often associated with a) cause unknown; b) hypertension, diabetes mellitus or chronic open angle glaucoma.

Since there were subtle bilateral signs, the patient was evaluated for other conditions such as serum viscosity syndromes, blood dyscrasias, clotting deficiencies, other real radiologic disorders, myeloproliferative disease, retinal vasculitis, macroglobulinemias, cryoglobulinemias.

Laser photocoagulation should be performed for proliferative sequelae. It is not particularly effective for macular edema in CVO, but could be considered as could the more experimental retinal vein choroidal vessel laser induced anastomoses.

In summary the patient was diagnosed with CRVO in both eyes. Additionally, the patient's visual acuity was 20/200 uncorrected; 20/30+corrected; pinhole 20/20 for the patient's right eye and 20/400 uncorrected; 20/200 corrected; pinhole unimprovable for the patients left eye.

On May 14, 1999, the patient was examined again and it was noted that the patient had keratoconus bilaterally, diagnosed in the teenage years, treated with spectacles and then contact lenses. The patient had always had excellent corrected visual acuity that the patient found comparable bilaterally. The patient was aware that more scarring in the cornea of the right eye has developed. However, after changing contact lens correction during the late winter, the patient then noticed that the left eye was not seeing as well. The patient did not recognize this as an acute change, though sometime in February the patient recognized that a central scotoma had developed. Since this recognition, the scotoma seems to be somewhat smaller and less dense. The patient was diagnosed as having a central vein occlusion by one doctor who referred the patient to another doctor. The second doctor confirmed this diagnosis and suggested that the patient be seen in approximately six weeks, or two weeks from now. The father of the patient is believed to have had a similar retinal process. Otherwise, the patient was completely healthy and not using any medications.

The patient's corrected acuity was 20/32-OD and with pinhole 20/25, whereas the left eye was 4/200 not improving with pinhole. Pupillary exam definitely demonstrates an afferent defect in the left eye. Intraocular pressure was 14 OD and 13 OS prior to dilation. Slit lamp examination shows significant keratoconus, right eye more so than left eye, with stromal scarring just inferior to the visual axis in the right eye. No scarring was seen in the left eye. There was no rubeosis in either eye. Gonioscopy of the left eye fails to identify any PAS or NVA.

A dilated exam of the left eye only was performed on the patient (Note: a recent, i.e., three weeks ago, examination of the right eye revealed that the right retina was healthy).

Extended ophthalmoscopy including contact lens biomicroscopy suggested that the cup to disc ratio was less than 0.3. There were dense intraretinal hemorrhages throughout four quadrants, particularly dense posteriorly. Dilated and tortuous venous system was seen. Only one nerve fiber layer infarct was present and there was no neovascularization. The patient has severe hemorrhagic cystic macular edema.

Clinically the patient had a central vein occlusion which was likely of indeterminate perfusion if a fluorescein angiogram was performed. It would likely be indeterminate because of the density of the posterior hemorrhages. The patient had several risk factors of possible progression to more widespread retinal ischemia, including his age, gender, level of acuity, and relatively recent onset. Therefore, it was determined that the patient should be followed at four to six week intervals to look for anterior or posterior segment neovascularization. Should this occur then PRP would be recommended. Otherwise, there was probably no other roles for laser photocoagulation since it has not been found effective in improving visual outcomes in individuals over 65 who have persistent CME. The fact that this eye was very unlikely to recover significant amounts of vision have been discussed. Under 20% of patients who have this level of acuity recover vision greater than 20/160 over time. For these reasons it was even more important that the right eye be carefully monitored. The patient will continue to do visual tasks and the patient was encouraged to learn to play tennis with the reduced level of depth perception. There was no reason for the patient to cover the left eye, though the patient has been given permission to do so if the patient finds that it interferes with the visual information obtained through the patient's right eye.

Moreover, the doctor noted that there was no role for aspirin or other anticoagulants, as there was no definitive proof that this will affect the course of the disease and there was also concern that it might increase hemorrhage, particularly in the optic nerve, and possibly potentiate the obstruction.

To summarize the above, the second doctor concurred with the diagnosis of CRVO. The doctor stated that it was unlikely that the patient will recover any of the lost visual acuity. Furthermore, it was noted that the patients' corrected acuity was 20/32 and with pinhole 20/25 for the right eye, whereas the left eye was 4/200 not improving with pinhole.

On Sep. 24, 1999, the patient was examined. On this date, the doctor noted that the patient presented with central retinal vein occlusion, left eye, and venous tortuosity and engorgement, right eye on Apr. 12, 1999. The patient's visual acuity then was 20/30+ OD, 20/200 OS. The exudative changes at the left macula have increased; in fact, there was massive edema at the macula, but there was no neovascularization in this eye. The right fundus was unchanged. The patient was understandably distressed.

Because the condition was bilateral, a medical workup was performed, however, the data from his laboratory appeared noncontributory. The patient reported that the medical workup was negative.

On Dec. 20, 1999, the patient was examined yet again. It was noted that the patient reported that vision OS remained blurred but perhaps with some improvement. The right eye was asymptomatic.

It was further reported that examinations of the patient revealed visual acuity OD 20/30 with contact lens, 20/25 with pinhole; OS count fingers, unimprovable. Applanation tension was 16 OD, 14 OS. An afferent pupil defect was present OD. At the slit lamp, there was no sign of rubeosis iridis. The right fundus continues to display normal disc, macula, major vessels, media and periphery. For the left, there continued to be present massive elevated edema involving the optic nerve and posterior pole with extensive hemorrhagic changes. However, there was no neovascularization. The diagnosis was central retinal vein occlusion OS.

Of note, it was reported that no therapy at the time would be beneficial. The only good sign was that the condition had been present since April 1999 and there was no sign of progressive ischemic changes leading to neovascularization.

As shown above, the examination demonstrated that the patient had CRVO in left eye and further that no therapy would be beneficial. The best corrected visual acuity for the patient was 20/25 for the right eye and that the left eye was unimprovable.

On Mar. 16, 2000, the patient was examined. It was noted that since last December, the patient sought additional opinions at National Institutes of Health for the patient's very severe central retinal vein occlusion left eye, which has damaged the posterior pole considerably, and blood tests for genetics.

The patient was enthusiastic that they were concentrating on the good eye (the right eye) and performing a variety of electrophysiologic studies and to see if they can learn about a prognostic sign that would indicate whether this right eye would develop central retinal vein occlusion.

It was reported that the patient's acuity was 20/25+OD with contact lens, 5/200 OS, unimprovable with afferent pupil defect OS. The right fundus continued to display, as it did initially, mild retinal venous tortuosity and engorgement, but was otherwise within normal limits. The left displayed very, very extensive macula and posterior pole edema with scattered retinal infarctions and hemorrhages and edema along the major retinal vein branches. There was no neovascularization. At the slit lamp, the right eye displayed mild cortical changes, the left +2 nuclear sclerosis, but there was no rubeosis. The diagnosis was central retinal vein occlusion OS; minimal central retinal vein occlusion changes OD versus congenital tortuosity of retinal vessels.

In sum the patient had very severe central retinal vein occlusion left eye, which had damaged his posterior pole considerably. The patient's visual acuity was 20/25+ with contact lens for the right eye and 5/200, unimprovable for the left eye.

In April 2000, the patient received a prescription for Viagra™ in a 50 mg dosage for an unrelated medical condition. At this time, the patient began taking 50 mg of Viagra™ on a regular basis. Additionally, as of Apr. 18, 2002, the patient has continued to take Viagra™ on a regular basis. Of particular note and as described above, prior to administration of Viagra™, the patients CRVO condition as well as the patient's eyesight progressively declined. However, as described below, following a regular administration of 50 mg of Viagra™, the patient's CRVO condition stabilized and the patients eyesight improved.

On Oct. 13, 2000 the patient was examined and it was reported that the patient had an ischemic central retinal vein occlusion in the left eye and a non-ischemic central vein occlusion in the right eye. It was further reported that the patients vision in the right eye had improved.

Of note, a complete coagulation work-up done was unremarkable. Therefore, it was reported that there was no evidence, despite a potential family history of CRVO, of any systemic abnormality in this patient. The patient's best-corrected visual acuity was 20/32+2 in the right eye and 20/800 in the left eye. The patient's intraocular pressures were 10 in the right eye and 12 in the left eye. A slit lamp examination showed no evidence of any neovascularization on the iris in either eye and gonioscopy of the left eye showed angles of grade 1 360° without evidence of neovascularization.

Additionally, a dilated funduscopic examination of the right eye showed continual venial tortuosity without evidence of cystoid macular edema or neovascularization with some peripheral scattered drusen. There were some small shunt vessels on the disc. Dilated funduscopic examination of the left eye showed extensive changes throughout the retina with areas of lipid and fibrosis. On the disc there were also numerous shunt vessels not extending into the vitreous. Of note, there was an inferior area of periretinal hemorrhage with a few small areas of less than 500-micron neovascularization of the retina.

Moreover, the patient was in a stable state at the time and without any evidence of any progression in the right eye. These small areas of neovascularization of the retina in the left eye were to be watched and possibly the recommendation of laser photocoagulation only if there was demonstrated progression.

In sum, the patient exhibited no evidence of progression in the right eye and the patients best-corrected visual acuity was 20/32+2 in the right eye and 20/800 in the left eye.

On Nov. 28, 2000, the patient reported vision was slightly improved in the left eye.

On Feb. 8, 2001, the patient was examined again. It was reported that the patient returned having been followed in the past for central retinal vein occlusion left eye.

The patient's vision was 20/20 right eye and 1/200 left eye. The right fundus continued to display mild retinal venous engorgement and tortuosity, which could represent an early central retinal vein occlusion though the findings were chronic or congenital tortuosity. The left displayed more growth tortuosity of the major retinal vein branches with submacular fibrosis and scattered posterior pole lipid. There was no neovascularization. The other positive observation for the patient was right eye, small anterior corneal leukoma and a +2 APD OS.

The patient was stable and it was recommended that the patient return in six months.

On Aug. 2, 2001, the patient was examined again. The patient had central retinal vein occlusion, left eye for a very long time. The patient did receive other opinions and was being followed also at the NIH.

Upon examination of the patient, it was noted that the right eye had always displayed some mild venous tortuosity and slight engorgement, but this was not progressive through today. The patient reported a slight improvement in peripheral vision, left eye.

The patient's visual acuity, right eye, was 20/20 with contact lens; 1/200 left eye (uncorrected formerly). Applanation tension 12 OU. Anterior segments revealed no evidence of rubeosis. Fundus OD, as previously described, unchanged; OS mild venous tortuosity, slightly normal disc, residual posterior pole edema, and lipid with considerable resolution of these changes and the former massive intraretinal hemorrhage. The diagnosis was central retinal vein occlusion, left eye, resolving anatomically; congenital tortuosity of retinal vessels OD versus mild venous statis, stable.

As shown above, the patient's visual acuity was 20/20 corrected vision for the right eye and 1/200 uncorrected vision for the left eye. The doctor's diagnosis was that the patient's CRVO was resolving in the left eye. Additionally the patient exhibited no evidence of progression in the right eye.

While the improvement in sight of the patient did not correct the problem because of the advanced stage the Viagra was administered, if administration at an early stage, there are indications that it could arrest or reverse the occlusion.

It is contemplated that the dosages of sildenafil currently used for Viagra™ may provide a guideline for administration of Viagra™ as an agent for combating RVO disease and other age related diseases. Furthermore, symptoms of RVO treated by the administration of Viagra™ include, but are not limited to, blurred vision, eye pain, and loss of vision. Some variation in dosage will necessarily occur depending on the condition of the subject being treated. The person responsible for administration will, in any event, determine the appropriate dose for the individual subject.

One skilled in the art would appreciate that this treatment could extend to a variety of conditions similar to RVO, particularly ocular circulatory conditions. Ocular circulatory is meant to include both intraocular blood circulation and aqueous humor circulation.

We claim:

1. A method of treating a symptom of an ocular circulatory condition in a human being comprising administering to said human being an effective amount of 1-[4-ethoxy-3-(6,7-dihydro-1-methyl-7-oxo-3-propyl-1H-pyrazolo-[4,3-d]pyrimidin-5-yl)phenylsulphonyl]-4-methylpiperazine or a pharmaceutically acceptable salt thereof for treating said symptom of said ocular circulatory condition.

2. The method of treating a symptom of an ocular circulatory condition according to claim 1, wherein said acceptable salt is a citrate salt.

3. The method of treating a symptom of an ocular circulatory condition according to claim 1, wherein said 1-[4-ethoxy-3-(6,7-dihydro-1-methyl-7-oxo-3-propyl-1H-pyrazolo-[4,3-d]pyrimidin-5-yl)phenylsulphonyl]-4-methylpiperazine or a pharmaceutically acceptable salt thereof is administered orally.

4. The method of treating a symptom of an ocular circulatory condition according to claim 1, wherein said effective amount of 1-[4-ethoxy-3-(6,7-dihydro-1-methyl-7-oxo-3-propyl-1H-pyrazolo-[4,3-d]pyrimidin-5-yl)phenylsulphonyl]-4-methylpiperazine or a pharmaceutically acceptable salt thereof is about 25 to about 100 mg per day.

5. The method of treating a symptom of an ocular circulatory condition according to claim 1, wherein said ocular circulatory condition is retinal vein occlusion.

6. The method of treating a symptom of an ocular circulatory condition according to claim 5, wherein said symptom is at least one of blurred vision, eye pain, and loss of vision.

7. A method of treating retinal vein occlusion comprising administering a safe and effective amount of a pharmaceutical composition to a patient in need thereof, wherein said pharmaceutical composition is sildenafil; or a pharmaceutically acceptable salt thereof.

8. The method of treating retinal vein occlusion according to claim 7, wherein said pharmaceutically acceptable salt is a citrate salt.

9. The method of treating retinal vein occlusion according to claim 7, wherein said pharmaceutical composition is administered orally.

10. The method of treating retinal vein occlusion according to claim 7, wherein said safe and effective amount of said pharmaceutical composition is about 25 to about 100 mg per day.

11. A method of preventing the progression of retinal vein occlusion comprising administering a safe and effective amount of a pharmaceutical composition to a patient in need thereof, wherein said pharmaceutical composition is sildenafil; or a pharmaceutically acceptable salt thereof.

12. The method of preventing the progression of retinal vein occlusion according to claim 11, wherein said pharmaceutically acceptable salt is a citrate salt.

13. The method of preventing the progression of retinal vein occlusion according to claim 11, wherein said pharmaceutical composition is administered orally.

14. The method of preventing the progression of retinal vein occlusion according to claim 11, wherein said safe and effective amount of said pharmaceutical composition is about 25 to about 100 mg per day.

15. A method of preventing the progression of a symptom of retinal vein occlusion in a human being comprising administering to said human being an effective amount of 1-[4-ethoxy-3-(6,7-dihydro-1-methyl-7-oxo-3-propyl-1H-pyrazolo-[4,3-d]pyrimidin-5-yl)phenylsulphonyl]-4-methylpiperazine or a pharmaceutically acceptable salt thereof for treating said symptom of retinal vein occlusion.

16. The method of preventing the progression of a symptom of retinal vein occlusion according to claim 15, wherein said acceptable salt is a citrate salt.

17. The method of preventing the progression of a symptom of retinal vein occlusion according to claim 15, wherein said 1-[4-ethoxy-3-(6,7-dihydro-1-methyl-7-oxo-3-propyl-1 H-pyrazolo-[4,3-d]pyrimidin-5-yl)phenylsulphonyl]-4-methylpiperazine or a pharmaceutically acceptable salt thereof is administered orally.

18. The method of preventing the progression of a symptom of retinal vein occlusion according to claim 15, wherein said effective amount of 1-[4-ethoxy-3-(6,7-dihydro-1-methyl-7-oxo-3-propyl-1H-pyrazolo-[4,3-d]pyrimidin-5-yl)phenylsulphonyl]-4-methylpiperazine or a pharmaceutically acceptable salt thereof is about 25 to about 100 mg per day.

19. The method of preventing the progression of a symptom of retinal vein occlusion according to claim 15, wherein said symptom is at least one of blurred vision, eye pain, and loss of vision.
